(19) United States
(12) Patent Application Publication
(10) Pub. No.: US 2003/0134796 A1
(43) Pub. Date: Jul. 17, 2003

(54) Antithrombin for the prevention and therapy of vasculoproliferative disorders

(76) Inventors: Juergen Roemisch, Vosendorf (AT); Harald Stauss, Dautphetal (DE); Hans Hoelschermann, Pohlheim (DE)

Correspondence Address: Finnegan, Henderson, Farabow,, Garrett & Dunner, L.L.P., 1300 I Street, N.W., Washington, DC, 20005-3315, US

(21) Appl. No.: 10/309,356

(22) Filed: Dec. 4, 2002

(30) Foreign Application Priority Data
Dec. 5, 2001 (DE) 101 59 556.5

(51) Int. Cl. A61K 38/36

(52) U.S. Cl. 514/12

(57) ABSTRACT

Antithrombin, in particular antithrombin III, is an agent or active ingredient suitable for the prevention and/or therapy of vasculoproliferative disorders such as, for example, transplant vasculopathy, restenosis, in-stent restenosis, and pulmonary hypertension.

The present invention relates to the use of antithrombin (AT), and in particular the use of antithrombin III (AT III), for the prevention and/or therapy of vasculoproliferative disorders. The present invention additionally encompasses corresponding medicaments and processes for the production thereof.

Vasculoproliferative disorders mean, for the purposes of the present invention, preferably transplant vasculopathy, restenosis, in-stent restenosis, and pulmonary hypertension. However, the present invention also encompasses the prevention and/or therapy of other disorders involving a proliferation of vessel walls that are harmful for the patient.

Transplant vasculopathy (TVP) is a form of coronary heart disease which affects the entire coronary vascular bed in the transplanted heart. It can lead to rapid occlusions of vessels, and represents the commonest cause of death in the first postoperative year for patients who have undergone a heart transplant. Similar vessel changes are observed in the coronary vascular bed following balloon angioplasty and stent implantation, and in the pulmonary vascular bed in patients with pulmonary hypertension.

Chronic endothelial damage caused by immunological and non-immunological mechanisms is thought to be responsible for coronary myointimal proliferation after heart transplants (TVP) and eventually results in migration and proliferation of smooth muscle cells within the entire coronary vascular system (see, for example, Circulation 96:2069,1997). Despite distinct advances in the areas of immunosuppression, of treatment of rejection, of organ preservation, and of management of critical infections, the incidence and severity of transplant vasculopathy following transplantations are undiminished.

These facts suggest that there are as yet unknown causal factors in the pathogenesis of this disorder. There is evidence that intravascular hypercoagulability with the occurrence of thrombosis and fibrin deposits in the vascular bed of the transplanted heart might, together with inflammatory/immunological damage, contribute to the intimal proliferation in this pathological state. It has been possible to demonstrate latent activation of the hemostasis system in patients who have undergone a heart transplant (see, for example, Circulation 96:2302, 1997; Blood 97:1079, 2001).

However, many possible factors and their interaction, which are still unknown in this connection, must be considered as a possible cause of such an activation of coagulation in patients who have undergone transplants. Thus, for example, local activation of coagulation in the vascular bed might take place through the release of a large number of atherogenic mediators such as, for example, tissue factor, thrombin, fibrin, platelet-derived growth factor, basic fibroblast growth factor, and others that induce the migration and proliferation of smooth muscle cells and thus might be significant for the process of myointimal proliferation (Crit. Care Med. 29:134, 2001; Int. Care Med. 26:704, 2000; Circulation 96:4232, 1997).

However, transplant vasculopathy is not a problem specific to the heart in transplant surgery. Identical proliferations of vessel walls also develop following transplantation of other organs, especially lung, kidney, liver, and pancreas, and limit the function and survival of the transplant in these cases too. Myointimal proliferation thus appears to be an immunological/inflammatory response of the body to a donor organ. Successful prevention and therapy of TVP ought therefore to be accompanied by prophylaxis of transplant rejection.

The harmful proliferation of vessel walls described in connection with transplant vasculopathy also influences the pathological state of primary and secondary pulmonary hypertension, and such proliferative changes in vessels are also observed in patients in relation to the development of restenosis after balloon angioplasty or stent implantation.

Treatment with the anticoagulant heparin in animal experiments only partially inhibited the proliferation of vessel walls (intimal proliferation) after a heart transplant, however (see, for example, Circulation 96:4232, 1997). The use of antithrombotics in the above-mentioned cases evidently does not show the desired effect.

Thus, at present, no suitable protective or therapeutic treatment procedures are available for such vasculoproliferative disorders. It was thus an object of the present invention to provide treatment procedures and pharmaceutical compositions suitable for these cases.

It has been found, surprisingly, that antithrombin, especially antithrombin III, can be used for the prevention and therapy of vasculoproliferative disorders, preferably of the disorders mentioned herein, and of the intimal damage and myointimal proliferation associated therewith.

Accordingly, the invention provides a method of treating a subject having, or suspected of having or developing, a vasculoproliferative disorder. It likewise provides a method of treating a subject having, or suspected of developing, the intimal damage and/or myointimal proliferation associated with a vasculoproliferative disorder. The method comprises administering an antithrombin to a subject in an amount sufficient to treat the vasculoproliferative disorder, the intimal damage, the myointimal proliferation, or a combination of two or more of these disorders and damage. Amounts to administer to each individual subject can be determined by those of skill in the art without undue or excessive experimentation based on standard protocols and regimens known in the art.

The invention additionally encompasses the use of an antithrombin, such as AT III, for preventing transplant rejection by the recipient organism. The method comprises administering to a subject an antithrombin in an amount sufficient to prevent rejection of the transplanted organ. Amounts to administer to each individual subject can be determined by those of skill in the art without undue or excessive experimentation based on standard protocols and regimens known in the art.

Antithrombin III (AT III) is a proteinase inhibitor which, besides thrombin, also inhibits other proteases such as kallikrein and the plasma coagulation factors IXa, Xa and XIIa. Besides AT III, other antithrombin forms are also distinguished, for example, AT I-AT VI. Antithrombin means, in general, the plasma activity directed against thrombin.

Anti thrombin/antithrombin III and methods for obtaining it, (for example from plasma), are known and described in the literature. In addition, antithrombin III is obtainable commercially, such as from pharmacies.

The present invention thus also encompasses medicaments comprising antithrombin and/or antithrombin III. Such medicaments can comprise typical components/ingredients used in medicaments, such as carriers, solvents, fillers, excipients, etc. In the medicament, the antithrombin (such as antithrombin III) is an active agent or ingredient.

The present invention is explained in detail by the following example:

The antiproliferative effect of AT III and thus its suitability for the prevention and/or therapy of vasculoproliferative disorders has been demonstrated by the present inventors in animal experiments on heterotopic, allogeneic heart transplantation in rats. This is an established animal model of transplant vasculopathy in which the transplants develop the same vascular lesions as following human heart transplants. The results obtained in this way make it possible to draw direct conclusions concerning corresponding use in humans.

The transplanted rats were treated in the first 14 days after the transplantation with antithrombin III in a dosage of 500 IU/kg of body weight, or placebo. All the animals received a standard immunosuppression with cyclosporin. 120 days after the transplantation, the severity of the transplant vasculopathy was significantly less for the AT-treated animals than in the placebo group. The severity of the coronary stenosis was, in this case, determined in histological sections of the transplanted hearts after an HE/elastica stain, and graded by means of a standardized scoring system into 4 degrees of severity. In this case, antithrombin III reduced both the incidence of transplant vasculopathy (in the AT-treated transplants there were distinctly more vessels without intimal proliferation) and the extent of the proliferation of vessel walls (in the AT-treated transplants the vessel stenosis was significantly less).

It will be apparent to those skilled in the art that various modifications and variations can be made in the practice of the present invention and without departing from the scope or spirit of the invention. Thus, other embodiments of the invention will be apparent to those skilled in the art from consideration of the specification and practice of the invention disclosed herein. It is intended that the specification and example be considered as exemplary only, with the true scope and spirit of the invention being indicated by the following claims.

Patent claims

1. The use of antithrombin, in particular antithrombin III, for the prevention and/or therapy of vasculoproliferative disorders.

2. The use as claimed in claim 1 for the prevention and/or therapy of transplant vasculopathy.

3. The use as claimed in claim 1 for the prevention and/or therapy of restenosis.

4. The use as claimed in claim 1 for the prevention and/or therapy of in-stent restenosis.

5. The use as claimed in claim 1 for the prevention and/or therapy of pulmonary hypertension.

6. A medicament comprising antithrombin, in particular antithrombin III.

7. A method for treating a vasculoproliferative disorder, said method comprising administering to a subject an antithrombin in an amount sufficient to treat said vasculoproliferative disorder.

8. The method of claim 7, wherein the antithrombin is antithrombin III (AT III).

9. The method of claim 7, wherein the method is a method of therapy.

10. The method of claim 7, wherein the method is a method of prevention.

11. The method of claim 7, wherein the method treats transplant vasculopathy.

12. The method of claim 7, wherein the method treats restenosis.

13. The method of claim 12, wherein the restenosis is in-stent restenosis.

14. The method of claim 12, wherein the restenosis occurs after balloon angioplasty.

15. The method of claim 7, wherein the method treats pulmonary hypertension.

16. The method of claim 7, wherein the method treats disorders involving a proliferation of vessel walls.

17. The method of claim 7, wherein the method treats intimal damage, myointimal proliferation, or both.

18. A method for preventing organ transplant rejection, said method comprising administering to a subject an antithrombin in an amount sufficient to prevent rejection of said organ.

19. The method of claim 18, wherein the organ is a heart.

20. The method of claim 18, wherein the organ is a lung.

21. The method of claim 18, wherein the organ is a kidney.

22. The method of claim 18, wherein the organ is a liver.

23. The method of claim 18, wherein the organ is a pancreas.

24. The method of claim 18, wherein the method further comprises treating with an immunosuppressant.

25. The method of claim 24, wherein the immunosuppressant is cyclosporin.
